(19) United States
(12) Patent Application Publication
(10) Pub. No.: US 2019/0240062 A1
(43) Pub. Date: Aug. 8, 2019

(54) Triangular urinary athletic cup

(71) Applicant: Karen S. Pinales, El Paso, TX (US)

(72) Inventor: Karen S. Pinales, El Paso, TX (US)

(21) Appl. No.: 15/932,087

(22) Filed: Feb. 2, 2018

(51) Int. Cl. A61F 5/453

(52) U.S. Cl. CPC A61F 5/453

(57) ABSTRACT

The present invention comprises a novel Triangular Urinary Athletic Cup (TUAC) incontinence prosthetic product with a unique design. The TUAC incontinence prosthetic product fits under regular underwear garments and generally consists of two parts.
1) The main collection portion, the cup, made of soft silicone, hard plastic, soft plastic or a combination thereof. This portion of the device is the flexible part.
2) A very rigid lower portion fastener attached to collection cup

### CROSS-REFERENCE TO RELATED APPLICATIONS

Provisional application no. 62/603,898, filed on Jun. 16, 2017

### STATEMENT REGARDING FEDERALLY SPONSORED RESEARCH OR DEVELOPMENT

Not applicable

### REFERENCE TO SEQUENCE LISTING A TABLE, OR A COMPUTER PROGRAM LISTING COMPACT DISC APPENDIX

Not applicable

### BACKGROUND OF THE INVENTION

The present invention relates to urinary incontinence prosthetic products. More specifically, the invention relates to male urinary incontinence products capable of being used without the need for incontinence diapers.

Urinary incontinence is the involuntary leakage of urine. Control over the urinary sphincter is either lost or weakened as in the cases involving the treatment of prostate cancer.

Certain other urinary incontinence prosthetic products and devices have been developed for the treatment of urinary incontinence. These devices for the most part, employ the use of straps and other bulky material which hinder the effective use of such products as they are uncomfortable for the user to wear long-term.

Therefore, a need exists in the field for novel incontinence products capable of efficiently treating incontinence without the need of bulky materials which is important for ease of movement and comfort in continued use.

### BRIEF SUMMARY OF THE INVENTION

The present invention comprises a novel Triangular Urinary Athletic Cup (TUAC) incontinence prosthetic product made of silicone with a unique design. The TUAC incontinence prosthetic product fits under regular underwear garments and generally consists of two parts.

1) The main collection portion, the cup, made of soft silicone. This portion of the device is the flexible part.
2) A very rigid lower fastener attached to collection cup

### BRIEF DESCRIPTION OF THE SEVERAL VIEWS OF THE DRAWINGS

The present disclosure is to be considered as an exemplification of the invention and is not intended to limit the invention to the specific embodiments illustrated by the figures or description below.

FIG. 1 is a front and bottom view along with perspective and top/sides view of a urinary incontinence prosthetic product showing my new design; The TUAC consists of two parts. The collection cup and a rigid fastener. The collection cup 1 is made of Silicone or other similar or suitable materials which produce suitable flexibility. The collection cup is of assorted sizes to fit a range of users. The rigid fastener 2 may be made from hard plastic or other suitable material.

FIG. 2 is a front and bottom view along with perspective and top/sides view of the collection cup configured with two apertures, a bodily aperture 3 and a fastener aperture 4. The bodily aperture is configured/sized to fit users of assorted sizes. The bodily aperture may be padded or unpadded. The fastener aperture may be a bolt type, screw type, stud type, or any other suitable fastener.

FIG. 3 is a front and bottom view along with perspective and top/sides view of the fastener which embodies two apertures. Aperture 5 is configured to attach to collection cup while, aperture 6 is configured to attach to a urine hose. The fastener may be made from hard plastic, soft plastic, a combination of any other suitable materials.

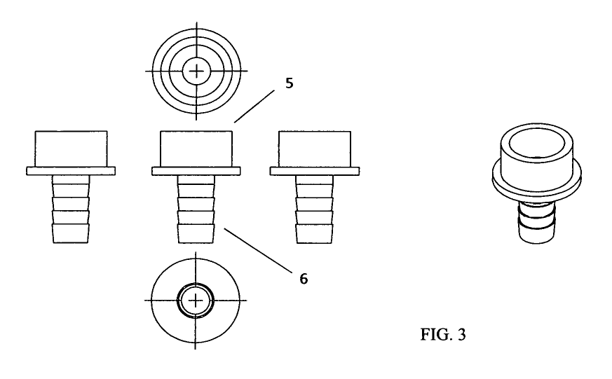

### DETAILED DESCRIPTION OF THE INVENTION

The terminology of the invention used herein is for describing embodiments only and is not intended to be limiting of the invention. It will be further understood that the terms “comprises” or “comprising” specify the presence of stated features, steps, operations, elements and/or groups thereof.

For the sake of clarity, this description will refrain from repeating every possible combination of the individual steps and the specification should be read with the understanding that such combinations are entirely within the scope of the invention and the claims.

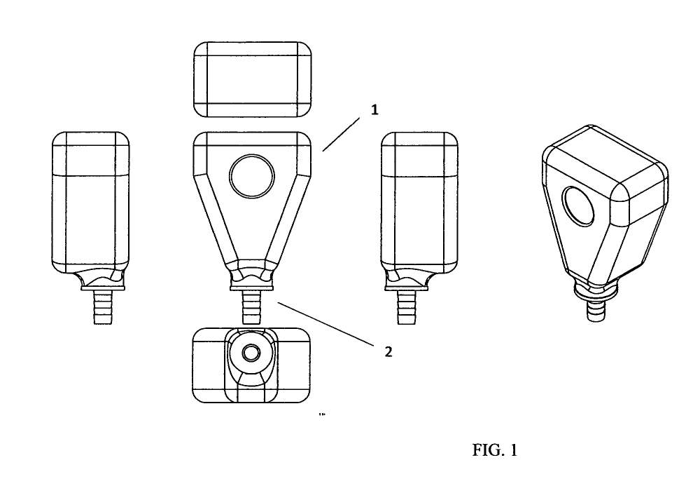

The elements of the device consist of two parts as shown in FIG. 1. The collection cup and a rigid fastener. The collection cup is made of Silicone, hard plastic, soft plastic, a combination thereof or any other similar or suitable materials which produce suitable flexibility for maximum comfort. The collection cup is of assorted sizes to fit a range of users. The rigid fastener may be made from hard plastic or other suitable material.

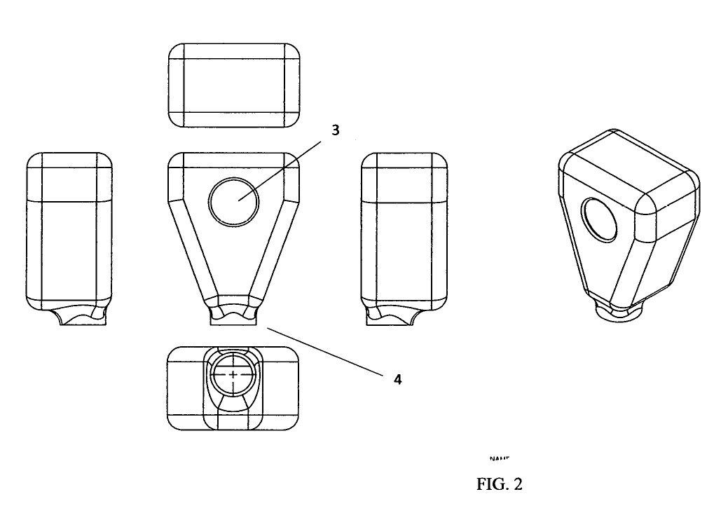

The collection cup in FIG. 2 is configured with two apertures, a bodily aperture and a fastener aperture. The bodily aperture is configured/sized to fit users of assorted sizes. The bodily aperture may be padded or unpadded. The fastener aperture may be a bolt type, screw type, stud type, or any other suitable fastener.

The fastener embodies two apertures. Aperture 3 is configured to attach to the collection cup while, Aperture 4 is designed to attach to any suitable urine hose currently in the market. The fastener may be made from hard plastic, soft plastic, or a combination of any other suitable materials.

Although the present invention has been illustrated and described herein with reference to preferred embodiments and specific examples thereof, it will be clear to those of ordinary skill in the art that other embodiments and examples may perform similar functions and/or achieve like results. All such equivalent embodiments and examples are within the spirit and scope of the present invention, are contemplated thereby, and are intended to be covered by the following claims.

What is claimed is:

1) The unique triangular design for a male urinary incontinence prosthetic product.

2) The two parts associated with said design; the collection cup and rigid fastener.
